(19) United States
(12) Patent Application Publication
(10) Pub. No.: US 2026/0188489 A1
(43) Pub. Date: Jul. 2, 2026

(54) SYSTEMIC BIOMARKERS FOR THE DYNAMIC ASSESSMENT OF TUMOR BURDEN AND TREATMENT EFFICACY IN HIGH GRADE GLIOMAS

(71) Applicants: TRUSTEES OF DARTMOUTH COLLEGE, Hanover, NH (US); MARY HITCHCOCK MEMORIAL HOSPITAL, FOR ITSELF AND ON BEHALF OF DARTMOUTH- HITCHCOCK CLINIC, Lebanon, NH (US)

(72) Inventor: Arti Gaur, Hanover, NH (US)

(73) Assignees: TRUSTEES OF DARTMOUTH COLLEGE, Hanover, NH (US); MARY HITCHCOCK MEMORIAL HOSPITAL, FOR ITSELF AND ON BEHALF OF DARTMOUTH- HITCHCOCK CLINIC, Lebanon, NH (US)

(21) Appl. No.: 19/130,009

(22) PCT Filed: Nov. 14, 2023
(86) PCT No.: PCT/US2023/037267

§ 371 Date: May 14, 2025

Related U.S. Application Data
(60) Provisional application No. 63/425,012, filed on Nov. 14, 2022.

(51) Int. Cl. G16H 50/20; C12Q 1/6809; C12Q 1/6886; G01N 33/68; G16H 10/60; G16H 20/10; G16H 50/70

(52) U.S. Cl. CPC G16H 50/20; C12Q 1/6809; C12Q 1/6886; G01N 33/6866; G01N 33/6869; G16H 10/60; G16H 20/10; G16H 50/70; C12Q 2600/118; C12Q 2600/178; G01N 2333/485; G01N 2333/522; G01N 2333/523; G01N 2333/535; G01N 2333/5434; G01N 2333/5443; G01N 2333/56

(57) ABSTRACT

Embodiments of the present disclosure pertain to methods of assessing glioma in a subject by (1) receiving a plurality of measured biomarker levels of the subject; and (2) assessing glioma in the subject based on the measured biomarker levels by at least correlating differentially expressed levels of the measured biomarkers to at least one of (a) diagnosis of glioma, (b) progression of glioma, (c) treatment outcome, and/or (d) tumor burden. The methods of the present disclosure also include a step of (3) making a treatment decision based on the assessment. Additional embodiments of the present disclosure pertain to systems for assessing glioma in a subject.

### CROSS-REFERENCE TO RELATED APPLICATIONS

This application claims priority to U.S. Provisional Patent Application No. 63/425,012 , filed on Nov. 14, 2022. The entirety of the aforementioned application is incorporated herein by reference.

### STATEMENT REGARDING FEDERALLY SPONSORED RESEARCH

This invention was made with government support under 1UG1CA189823-01 awarded by the National Institutes of Health. The government has certain rights in the invention.

### BACKGROUND

Current methods of detecting, monitoring and treating gliomas suffer from numerous limitations. Various embodiments of the present disclosure address the aforementioned limitations.

### SUMMARY

In some embodiments, the present disclosure pertains to methods of assessing glioma in a subject. In some embodiments, the methods of the present disclosure include: (1) receiving a plurality of measured biomarker levels of the subject; and (2) assessing glioma in the subject based on the measured biomarker levels by at least correlating differentially expressed levels of the measured biomarkers to at least one of (a) diagnosis of glioma, (b) progression of glioma, (c) treatment outcome, and/or (d) tumor burden. In some embodiments, the methods of the present disclosure also include a step of (3) making a treatment decision based on the assessment. In some embodiments, the treatment decision includes (a) monitoring the course of the glioma, (b) removing the glioma from the subject, (c) administering a therapeutic agent to the subject, and/or (d) modifying a pre-existing treatment regimen. In some embodiments, the methods of the present disclosure may be repeated after implementing the treatment decision.

Additional embodiments of the present disclosure pertain to systems for assessing glioma in a subject. In some embodiments, the systems of the present disclosure include a computing device. In some embodiments, the computing device includes: (1) programming instructions for receiving a plurality of measured biomarker levels of the subject; and (2) programming instructions for assessing glioma in the subject based on the measured biomarker levels, where the assessment includes correlating differentially expressed levels of the measured biomarkers to at least one of (b) diagnosis of glioma, (b) progression of glioma, (c) treatment outcome, and/or (d) tumor burden. In some embodiments, the computing devices of the present disclosure also include (3) programming instructions for making a treatment decision based on the assessment.

### BRIEF DESCRIPTION OF THE DRAWINGS

FIGS. 1A-1D show magnetic resonance imaging (MRI) of pseudo-progression in glioblastoma (GBM) patients.

FIG. 2A provides an illustration of a method of assessing glioma in a subject in accordance with various embodiments of the present disclosure.

FIG. 2B provides an architecture of a computing device for assessing glioma in a subject in accordance with various embodiments of the present disclosure.

FIG. 3 provides time points for blood draws of consented GBM patients.

FIGS. 4A-4B demonstrate that specific systemic micro RNA (miRNAs) correlate with tumor burden and recurrence in GBM patients. FIG. 4A shows miRNA levels in plasma collected pre vs. post resection (Draw 1 vs. Draw 2). FIG. 4B shows miRNA levels in plasma collected post resection vs. progression (Draw 2 vs. Draw 6).

FIGS. 5A-5C provide additional data demonstrating that specific miRNAs correlate with tumor burden and recurrence in GBM patients. FIG. 5A shows miRNA levels in plasma collected from patients with pseudo progression vs. progression. FIG. 5B shows an MRI scan of a low tumor burden brain (pseudo progression). FIG. 5C shows an MRI scan of a high tumor burden brain (real tumor progression).

FIG. 6 shows a decrease in miRNA levels with treatment induced toxicities.

FIG. 7A shows concentrations of secreted proteins in pre-resection plasma from GBM patients vs. plasma from healthy donors.

FIGS. 7B-7C8 show concentrations of secreted proteins in pre-resection plasma from GBM patients vs. plasma taken immediately post tumor resection but before starting chemotherapy and/or radiation therapy. FIG. 7B demonstrates that glioma tumor burden correlates to decreased concentrations of circulating CCL2 and CCL22. FIG. 7C1-7C8 demonstrates that glioma tumor burden correlates to increased circulating G-CSF, IFNα2, EGF, IL-12p70, VEGF, IL-12p40, IL-15, and TNFα.

FIGS. 8A-8B show expression levels of miRNA panels. FIG. 8A shows the hierarchical clustering of the robust eleven microRNAs and their expression levels in a post resection draw (D2) and a post progression draw (DProg). Note the circled cluster of the 5 progression draws. These columns include four out of five DProg draws grouped together hierarchically, with 37D2 also present with them, appearing to go against the general trend of the rest of the data. FIG. 8B depicts the hierarchical clustering of D1 vs D2 with the specific eleven miRNA panel. Noticeable similarities are present in this panel shown through the clustering of three D2s and four D1s together.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein arc for organizational purposes and are not to be constructed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Malignant gliomas develop from the glial lineage within the central nervous system (CNS) and account for approximately 70% of the primary brain tumors diagnosed in the U.S. per year. Based on histology, the World Health Organization (WHO) classifies gliomas by grade: Grades I & II are low grade, while III and IV are high grade. Glioblastomas (GBM) are grade IV and are the most common malignant gliomas.

Gliomas, including glioblastomas (GBM), present a public health concern. In fact, gliomas make up 81% of malignant brain tumors in adults. In particular, GBMs are aggressive primary brain tumors that are diagnosed by histologic evaluation of tumor tissue. They are resistant to all available modalities of treatment with a median survival of only 14 months and a 5-year survival rate of less than 5%. These lethal tumors recur in all patients and the median overall survival of patients is 14.6 months.

Moreover, most GBMs are diagnosed only after symptom onset, via histological examination of resected tumors. However, GBMs harbor diverse oncogenes and mutated tumor suppressor genes with both inter-and intra-tumor variability in expression patterns, making their standardized molecular characterization impossible. For instance, GBMs harbor diverse oncogenes and mutated tumor suppressor genes, such as p53 mutations, deletion of PTEN and p16INK4A/p14ARF, EGF receptor amplification, and the activation of RAS-MAPK and PI3K-AKT signaling pathways, IDH mutations and changes in methylation of MGMT. The resulting cellular and molecular variability both at inter-and intra-tumor levels makes standardized histological characterization of GBM difficult.

Mortality rates in GBM patients have changed only slightly over the past 30 years, with median overall survival increasing by 3.3 months; from 11.3 months to 14.6 months. This is primarily due to the Standard of Care (SOC) established, where survival improved when daily temozolomide (TMZ) was administered with 6 weeks of radiation therapy (RT) and for 6 months thereafter. TMZ is a methylating agent and patients with methylated O-6-methylguanine-DNA methyltransferase (MGMT) as well as IDH-mutated GBM tumors respond better to RT and TMZ.

Despite the survival benefits, RT/TMZ can cause severe adverse side effects as well as progressive blood-brain barrier dysfunction that can result in pseudo progression which is clinical and radiologic deterioration without true tumor progression. About 15 to 20% of newly diagnosed patients receiving TMZ and RT develop prolonged grade 3-4 thrombocytopenia and a significant risk fatal myelosuppression. This limits further chemotherapy, necessitates frequent transfusions, and places patients at long-term risk of bleeding. Furthermore, around 98% of patients face a recurrence after a progression free survival (PFS) period of roughly 7 to 10 months.

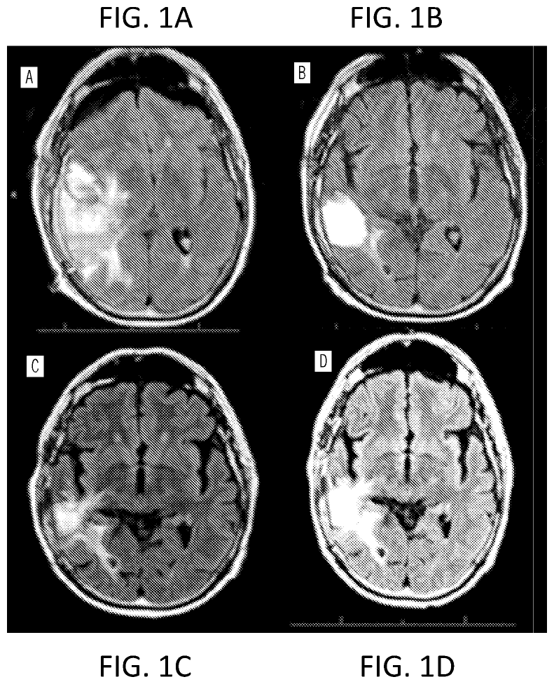

Neurological examination as well as magnetic resonance imaging (MRI) are the only methods for disease evaluation. However, MRIs present diagnostic challenges. An example of one of the diagnostic challenges where MRI characteristics falsely mimic tumor progression is shown in FIGS. 1A-1D of a patient. FIG. 1A is an MRI of the patient prior to surgical resection of primary tumor while FIG. 1B is a post-resection image 5 months after surgery. FIG. 1C is an image 9 months into treatment and shows new enhancing lesions that led to a second resection (FIG. 1D) due to the appearance of tumor growth on MRI. However, in these lesions, post-surgical resection were confirmed by the surgical neuropathologist to be RT induced necrosis due to hypo perfusion and not tumor growth.

Hence, there is a critical need to establish systems and methods to continuously assess treatment efficacy and toxicity while tracking tumor burden pre-and post-surgical resection. Numerous embodiments of the present disclosure aim to address this unmet need through the discovery of GBM-specific biomarkers that could allow for precise monitoring of GBM patients.

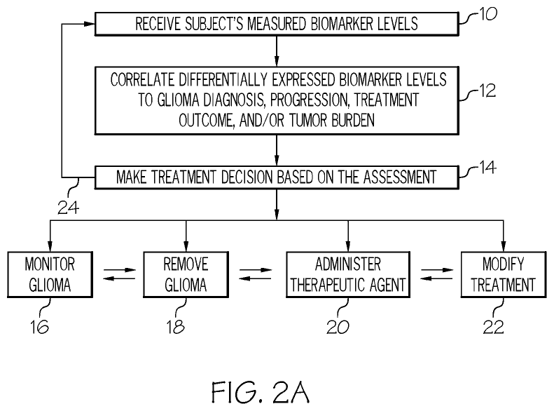

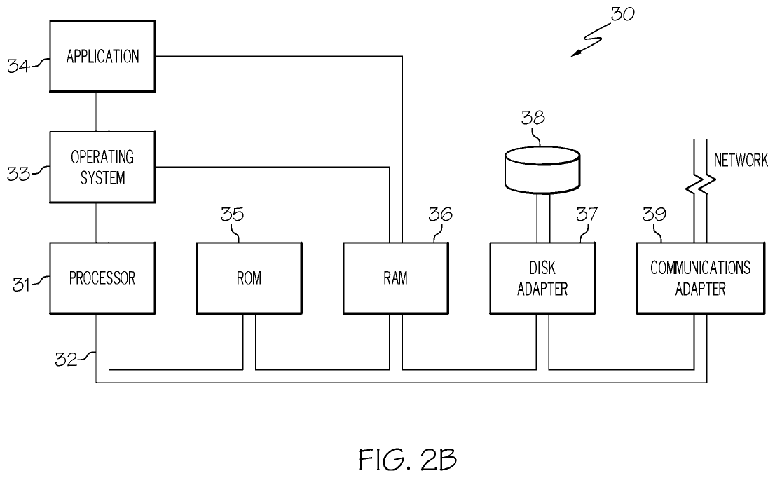

In some embodiments, the present disclosure pertains to methods of assessing glioma in a subject. In some embodiments illustrated in FIG. 2A, the methods of the present disclosure include: receiving a plurality of measured biomarker levels (e.g., systemic biomarker levels) of the subject (step 10); and assessing glioma in the subject based on the measured biomarker levels by at least correlating differentially expressed levels of the measured biomarkers to at least one of diagnosis of glioma, progression of glioma, treatment outcome, and/or tumor burden (step 12). In some embodiments, the methods of the present disclosure also include a step of making a treatment decision based on the assessment (step 14). For instance, in some embodiments, the treatment decision includes monitoring the course of the glioma (step 16), removing (e.g., surgically removing) the glioma from the subject (step 18), administering a therapeutic agent to the subject (step 20), modifying a pre-existing treatment regimen (step 22), or combinations thereof. In some embodiments, the methods of the present disclosure may be repeated after implementing the treatment decision (step 24).

Additional embodiments of the present disclosure pertain to systems for assessing glioma in a subject. In some embodiments, the systems of the present disclosure include a computing device. In some embodiments, the computing device includes: (1) programming instructions for receiving a plurality of measured biomarker levels of the subject; and (2) programming instructions for assessing glioma in the subject based on the measured biomarker levels, where the assessment includes correlating differentially expressed levels of the measured biomarkers to at least one of diagnosis of glioma, progression of glioma, treatment outcome, tumor burden, or combinations thereof. In some embodiments, the computing devices of the present disclosure also include (3) programming instructions for making a treatment decision based on the assessment. As set forth in more detail herein, the methods and systems of the present disclosure can have numerous embodiments.

### Assessment of Glioma

The methods and systems of the present disclosure may be utilized to assess various types of gliomas. For instance, in some embodiments, the glioma includes glioblastoma (GBM).

Additionally, the methods and systems of the present disclosure may assess glioma in a subject in various manners. For instance, in some embodiments, glioma assessment includes correlating differentially expressed levels of biomarkers to the diagnosis of glioma in the subject. In some embodiments, an increase in the levels of biomarkers is correlated to positive diagnosis of glioma. In some embodiments, a decrease in the levels of biomarkers is correlated to a negative diagnosis of glioma.

In some embodiments, glioma assessment includes correlating differentially expressed levels of the biomarkers to the progression of glioma in the subject. In some embodiments, the progression of glioma is characterized by at least one of an onset of tumors, tumor recurrence, survival prospects, or combinations thereof. In some embodiments, an increase in the levels of biomarkers is correlated to a positive progression of glioma (e.g., tumor recurrence). In some embodiments, a decrease in the levels of biomarkers is correlated to a negative progression of glioma (e.g., remission).

In some embodiments, glioma assessment includes correlating differentially expressed levels of the biomarkers to treatment outcome. In some embodiments, the treatment outcome is characterized by incidence of treatment induced toxicities. In some embodiments, the treatment induced toxicities include at least one of hematologic toxicity, profound myelosuppression, fatal thrombocytopenia, leukopenia, or combinations thereof. In some embodiments, an increase in the levels of biomarkers is correlated to a negative treatment outcome. In some embodiments, a decrease in the levels of biomarkers is correlated to a positive treatment outcome.

In some embodiments, glioma assessment includes correlating differentially expressed levels of the biomarkers to tumor burden. In some embodiments, the tumor burden is characterized by at least one of malignancies, brain metastasis, the number of cancer cells, the size of a tumor, the amount of glioma in the subject, or combinations thereof. In some embodiments, an increase in the levels of biomarkers is correlated to a positive tumor burden. In some embodiments, a decrease in the levels of biomarkers is correlated to a negative tumor burden.

### Biomarkers

The methods and systems of the present disclosure may utilize various measured levels of biomarkers to assess glioma. For instance, in some embodiments, the measured biomarker levels include one or more measured microRNA levels, one or more measured mRNA levels, one or more measured protein levels, or combinations thereof. In some embodiments, the measured biomarker levels include one or more measured microRNA levels. In some embodiments, the measured biomarker levels include one or more measured protein levels. In some embodiments, the measured biomarker levels include one or more measured microRNA levels and one or more measured protein levels.

In some embodiments, the one or more measured microRNA levels include one or more measured levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p, hu-mir-196b-5p, hu-mir-151a-3p, or combinations thereof.

In some embodiments, the one or more measured microRNA levels include measured levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p. In some embodiments, differential expression of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p are correlated to at least one of diagnosis of glioma, progression of glioma, treatment outcome, and/or tumor burden. In some embodiments, differential expression of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p are correlated to a change in tumor burden.

In some embodiments, increased levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p are correlated to at least a positive diagnosis of glioma, a positive progression of glioma, a negative treatment outcome, and/or a positive tumor burden. In some embodiments, decreased levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p are correlated to at least a negative diagnosis of glioma, a negative progression of glioma, a positive treatment outcome, and/or a negative tumor burden.

In some embodiments, increased levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p may be observed in subjects suffering from glioma prior to tumor resection. In some embodiments, decreased levels of the same microRNAs may be observed in the same subjects after the tumor resection relative to the microRNA levels prior to tumor resection. In some embodiments, microRNA levels have a directional change in blood concentration in each glioma subject that matched with their individual tumor burden when comparisons were made between samples collected before (between one day up to a week) tumor resection and within two weeks after tumor resection but before starting chemotherapy and/or radiation therapy for each subject.

In some embodiments, differential expression of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p are correlated to a change in tumor burden in subjects after tumor resection but before tumor progression, tumor recurrence, and initiation of standard of care treatment. In some embodiments, increased levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p in subjects suffering from glioma after tumor resection may be correlated to a positive tumor burden (e.g., tumor progression). In some embodiments, decreased levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p in subjects suffering from glioma after tumor resection may be correlated to a negative tumor burden.

In some embodiments, increased levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p are correlated to a positive tumor burden in subjects suffering from glioma after tumor progression and/or recurrence, after tumor resection, and after initiation of standard of care treatment when compared to subjects with no tumor progression (i.e., pseudo-progression). In some embodiments, decreased levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p in subjects suffering from glioma after the glioma was resected are correlated to a negative tumor burden (i.e., pseudo-progression). In some embodiments, decreased levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p in subjects suffering from glioma after the glioma was resected are correlated to a negative tumor burden (i.e., pseudo-progression).

In some embodiments, the one or more measured microRNA levels include measured levels of hu-mir-126-3p, hu-mir-142-3p, hu-mir-223-3p, hu-mir-196b-5p, and hu-mir-151a-3p. In some embodiments, the one or more measured microRNA levels include measured levels of hu-mir-126-3p, hu-mir-mir-142-3p, and hu-mir-mir-223-3p. In some embodiments, increased levels of hu-mir-126-3p, hu-mir-mir-142-3p, and hu-mir-mir-223-3p are correlated to a positive diagnosis of glioma, a positive progression of glioma, a negative treatment outcome, a positive tumor burden, or combinations thereof. In some embodiments, decreased levels of hu-mir-126-3p, hu-mir-mir-142-3p, and hu-mir-mir-223-3p are correlated to a negative diagnosis of glioma, a negative progression of glioma, a positive treatment outcome, a negative tumor burden, or combinations thereof. In some embodiments, increased levels of hu-mir-126-3p, hu-mir-mir-142-3p, and hu-mir-mir-223-3p are correlated to a positive progression of glioma (e.g., tumor progression).

In some embodiments, the one or more measured microRNA levels include measured levels of hu-mir-151a-3p. In some embodiments, increased levels of hu-mir-151a-3p are correlated to a positive diagnosis of glioma, a positive progression of glioma, a negative treatment outcome, and/or a positive tumor burden. In some embodiments, decreased levels of hu-mir-151a-3p are correlated to a negative diagnosis of glioma, a negative progression of glioma, a positive treatment outcome, and/or a negative tumor burden. In some embodiments, decreased levels of hu-mir-151a-3p are linked to a negative treatment outcome (e.g., incidence of treatment induced toxicities). In some embodiments, decreased levels of hu-mir-151a-3p in subjects suffering from glioma and undergoing standard of care treatment are linked to a negative treatment outcome (e.g., incidence of treatment induced toxicities).

In some embodiments, the one or more measured protein levels include one or more measured levels of CCL11, CCL2, CCL22, EGF, FGF-2, CXCL1, G-CSF, GM-CSF, IFN-α2, IL-6, IL-10, IL-12p40, IL-12p70, IL-15, IL-1Rα, IL-1α, TNFα, PDGF-AA, VEGF, or combinations thereof. In some embodiments, the one or more measured protein levels include measured levels of CCL2, EGF, G-CSF, IFN-α2, IL-6, and IL-10. In some embodiments, the one or more measured protein levels include measured levels of CCL2, IFN-α2, G-CSF, and EGF.

In some embodiments, the one or more measured protein levels include CCL2, CCL11, CCL22 and GM-CSF. In some embodiments, increased levels of CCL2, CCL11, CCL22 and GM-CSF are correlated to at least a negative diagnosis of glioma, a negative progression of glioma, a positive treatment outcome, and/or a negative tumor burden. In some embodiments, decreased levels of CCL2, CCL11, CCL22 and GM-CSF are correlated to at least a positive diagnosis of glioma, a positive progression of glioma, a negative treatment outcome, and/or a positive tumor burden. In some embodiments, increased levels of CCL2, CCL11, CCL22 and GM-CSF in subjects suffering from glioma when compared between before the glioma was resected (highest tumor burden) and after tumor resection (lowest tumor burden) are correlated to a negative tumor burden.

In some embodiments, the one or more measured protein levels include measured levels of CCL2 and CCL22. In some embodiments, increased levels of CCL2 and CCL22 are correlated to a negative diagnosis of glioma, a negative progression of glioma, a positive treatment outcome, and/or a negative tumor burden. In some embodiments, decreased levels of CCL2 and CCL22 are correlated to a positive diagnosis of glioma, a positive progression of glioma, a negative treatment outcome, and/or a positive tumor burden.

In some embodiments, the one or more measured protein levels include IFN-α2, EGF, FGF-2, CXCL1, IL-12p40, IL-12p70, IL-15, IL-1Rα, IL-1α, TNFα, VEGF and G-CSF. In some embodiments, increased levels of IFN-α2, EGF, FGF-2, CXCL1, IL-12p40, IL-12p70, IL-15, IL-1Rα, IL-1α, TNFα, VEGF and G-CSF are correlated to at least a positive diagnosis of glioma, a positive progression of glioma, a negative treatment outcome, and/or a positive tumor burden. In some embodiments, decreased levels of IFN-α2, EGF, FGF-2, CXCL1, IL-12p40, IL-12p70, IL-15, IL-1Rα, IL-1α, TNFα, VEGF and G-CSF are correlated to at least a negative diagnosis of glioma, a negative progression of glioma, a positive treatment outcome, and/or a negative tumor burden. In some embodiments, increased levels of IFN-α2, EGF, FGF-2, CXCL1, IL-12p40, IL-12p70, IL-15, IL-1Rα, IL-1α, TNFα, VEGF and G-CSF in subjects suffering from glioma when compared between before the glioma was resected (highest tumor burden) and after tumor resection (lowest tumor burden) are linked to a positive tumor burden.

In some embodiments, the one or more measured protein levels include measured levels of G-CSF, IFNα2, EGF, IL-12p70, VEGF, IL-12p40, IL-15, and TNFα. In some embodiments, increased levels of G-CSF, IFNα2, EGF, IL-12p70, VEGF, IL-12p40, IL-15, and TNFα are correlated to a positive diagnosis of glioma, a positive progression of glioma, a negative treatment outcome, and/or a positive tumor burden. In some embodiments, decreased levels of G-CSF, IFNα2, EGF, IL-12p70, VEGF, IL-12p40, IL-15, and TNFα are correlated to a negative diagnosis of glioma, a negative progression of glioma, a positive treatment outcome, and/or a negative tumor burden.

In some embodiments, differentially expressed levels of measured biomarkers represent elevated levels of one or more of the measured biomarkers, depressed levels of one or more of the measured biomarkers, or combinations thereof.

In some embodiments, the differentially expressed levels of the measured biomarkers represent elevated levels of one or more of the measured biomarkers. In some embodiments, elevated levels of a biomarker represent biomarker levels that are at least 10% higher than the average biomarker levels of subjects that are not suffering from glioma. In some embodiments, elevated levels of a biomarker represent biomarker levels that are at least 15% higher than the average biomarker levels of subjects that are not suffering from glioma. In some embodiments, elevated levels of a biomarker represent biomarker levels that are at least 20% higher than the average biomarker levels of subjects that are not suffering from glioma. In some embodiments, elevated levels of a biomarker represent biomarker levels that are at least 25% higher than the average biomarker levels of subjects that are not suffering from glioma. In some embodiments, elevated levels of a biomarker represent biomarker levels that are at least 50% higher than the average biomarker levels of subjects that are not suffering from glioma.

In some embodiments, the differentially expressed levels of the measured biomarkers represent depressed levels of one or more of the measured biomarkers. In some embodiments, depressed levels of a biomarker represent biomarker levels that are at least 10% lower than the average biomarker levels of subjects that are not suffering from glioma. In some embodiments, depressed levels of a biomarker represent biomarker levels that are at least 15% lower than the average biomarker levels of subjects that are not suffering from glioma. In some embodiments, depressed levels of a biomarker represent biomarker levels that are at least 20% lower than the average biomarker levels of subjects that are not suffering from glioma. In some embodiments, depressed levels of a biomarker represent biomarker levels that are at least 25% lower than the average biomarker levels of subjects that are not suffering from glioma. In some embodiments, depressed levels of a biomarker represent biomarker levels that are at least 50% lower than the average biomarker levels of subjects that are not suffering from glioma.

In some embodiments, the differentially expressed levels of the one or more protein levels include differentially expressed mRNA levels of the one or more proteins, differentially expressed protein levels of the one or more proteins, or combinations thereof. In some embodiments, the differentially expressed levels of the one or more protein levels include differentially expressed mRNA levels of the one or more proteins.

The differentially expressed levels of the measured biomarkers may represent measured biomarker levels at various stages of glioma. For instance, in some embodiments, the differentially expressed levels of the measured biomarkers represent differentially expressed levels of measured biomarkers prior to glioma diagnosis, at the time of glioma diagnosis, prior to tumor resection, after tumor resection, after receiving treatment for glioma, after glioma diagnosis and glioma progression, after tumor resection and glioma progression, or combinations thereof. In some embodiments, the differentially expressed levels of the measured biomarkers represent differentially expressed levels of the measured biomarkers prior to tumor resection. In some embodiments, the differentially expressed levels of the measured biomarkers represent differentially expressed levels of the measured biomarkers after tumor resection. In some embodiments, the differentially expressed levels of the measured biomarkers represent differentially expressed levels of the measured biomarkers after tumor resection and glioma progression.

In some embodiments, the differentially expressed levels of the measured biomarkers represent differentially expressed levels of the measured biomarkers relative to normal levels of the measured biomarkers. In some embodiments, the differentially expressed levels of the measured biomarkers represent differentially expressed levels of the measured biomarkers in the subject after receiving treatment for glioma relative to the levels of measured biomarkers in the subject prior to receiving treatment for glioma. In some embodiments, the differentially expressed levels of the measured biomarkers represent differentially expressed levels of the measured biomarkers in a subject after glioma tumor resection relative to the levels of measured biomarkers in the subject prior to glioma tumor resection.

### Measurement of Biomarker Levels

Biomarker levels may be measured from various samples of a subject. For instance, in some embodiments, the biomarker levels are measured from a blood sample of the subject. In some embodiments, the biomarker levels are measured from a tissue sample of the subject. In some embodiments, the biomarker levels are measured from a body fluid of the subject.

In some embodiments, the methods and systems of the present disclosure also include a step of measuring biomarker levels from a biological sample of a subject. In some embodiments, the methods and systems of the present disclosure also include a step of obtaining a biological sample from the subject and measuring the biomarker levels from the biological sample.

In some embodiments, the biological sample includes at least one of a tissue sample, body fluid, blood sample, or combinations thereof. In some embodiments, the biological sample is obtained from a subject after the subject has received treatment for glioma.

### Subjects

The methods and systems of the present disclosure may be utilized to assess glioma in various subjects. For instance, in some embodiments, the subject is a human being. In some embodiments, the subject is suffering from glioma. In some embodiments, the subject is suffering from glioblastoma (GBM). In some embodiments, the subject has received treatment or is receiving treatment for glioma.

### Glioma Assessment

The methods and systems of the present disclosure may be utilized to assess glioma in subjects in various manners. For instance, in some embodiments, the assessment occurs manually. In some embodiments, the assessment occurs in real-time. In some embodiments, the assessment occurs continuously.

In some embodiments, the assessment occurs automatically through the utilization of an algorithm. In some embodiments, the algorithm is a machine learning algorithm trained on the measured biomarkers.

In some embodiments, machine learning algorithm is an L1-regularized logistic regression algorithm. In some embodiments, the algorithm is a machine learning algorithm that is trained on the measured biomarkers. In some embodiments, the machine learning algorithm includes supervised learning algorithms. In some embodiments, the supervised learning algorithms include nearest neighbor algorithms, naïve-Bayes algorithms, decision tree algorithms, linear regression algorithms, support vector machines, neural networks, convolutional neural networks, ensembles (e.g., random forests and gradient boosted decision trees), and combinations thereof.

Machine learning algorithms may be trained in various manners. For instance, in some embodiments, the training includes: (1) feeding a first set of measured biomarker levels into a machine learning algorithm, where the first set of measured biomarker levels are from one or more subjects that have glioma; (2) feeding a second set of measured biomarker levels into the machine learning algorithm, where the second set of measured biomarker levels are from one or more subjects that do not have glioma; and (3) training the machine learning algorithm to assess the glioma by comparing the first set of measured biomarker levels with the second set of measured biomarker levels.

### Treatment Decision

In some embodiments, the methods and systems of the present disclosure also include a step of, or programming instructions for, making a treatment decision based on a glioma assessment. In some embodiments, the treatment decision includes monitoring the course of the glioma, removing the glioma from the subject (e.g., surgically removing the glioma), administering a therapeutic agent to the subject (e.g., an established and/or an experimental therapeutic agent), modifying a pre-existing treatment regimen, or combinations thereof. In some embodiments, the treatment decision includes modifying a pre-existing treatment regimen. In some embodiments, the modification includes reducing a dosage on a medication, stopping a treatment, or combinations thereof.

In some embodiments, the treatment decision includes monitoring the course of the glioma. In some embodiments, the treatment decision includes administering a therapeutic agent to the subject. In some embodiments, the therapeutic agent includes a chemotherapeutic agent. In some embodiments, the therapeutic agent includes radiation.

In some embodiments, the methods and systems of the present disclosure may be repeated after implementing the treatment decision. In some embodiments, the methods and systems of the present disclosure may be utilized to continuously assess the glioma. For instance, in some embodiments, the methods of the present disclosure may be repeated after the removal of a glioma tumor to assess tumor burden after the removal. In some embodiments, the methods of the present disclosure may be repeated after the administration of a therapeutic agent to assess the efficacy of the therapeutic agent in treating glioma.

Computing Devices

The methods and systems of the present disclosure can be implemented through the utilization of various computing devices with various architectures. Similarly, the computing devices of the present disclosure can include various architectures.

For instance, in some embodiments, the computing devices of the present disclosure are in electrical communication with an algorithm of the present disclosure. In some embodiments, the computing devices of the present disclosure include a web-based program, an application-based program, or combinations thereof. In some embodiments, the step of receiving measured biomarker levels includes entering the measured biomarker levels into the computing device. In some embodiments, programming instructions for receiving the measured biomarker levels include programming instructions for entering the measured biomarker levels into the computing device.

In some embodiments, the computing device includes a keyboard for a user to navigate and choose between different risk prediction functions. In some embodiments, the computing device further includes a display screen for displaying outputs from an algorithm.

The computing devices of the present disclosure can include various types of computer-readable storage mediums. In some embodiments, the computer-readable storage mediums can be a tangible device that can retain and store instructions for use by an instruction execution device. In some embodiments, the computer-readable storage medium may include, without limitation, an electronic storage device, a magnetic storage device, an optical storage device, an electromagnetic storage device, a semiconductor storage device, and combinations thereof. A non-exhaustive list of more specific examples of suitable computer-readable storage medium includes, without limitation, a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, and combinations thereof.

A computer-readable storage medium, as used herein, is not to be construed as being transitory signals per se. Such transitory signals may be represented by radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer-readable program instructions described herein can be downloaded to respective computing/processing devices from a computer-readable storage medium or to an external computer or external storage device via a network, such as the Internet, a local area network, a wide area network and/or a wireless network. In some embodiments, the network may include copper transmission cables, optical transmission fibers, wireless transmission, routers, firewalls, switches, gateway computers and/or edge servers. In some embodiments, a network adapter card or network interface in each computing/processing device receives computer-readable program instructions from the network and forwards the computer-readable program instructions for storage in a computer-readable storage medium within the respective computing/processing device.

In some embodiments, computer-readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object-oriented programming language such as Smalltalk, C++, or the like, and procedural programming languages, such as the “C” programming language or similar programming languages.

In some embodiments, the computer-readable program instructions may execute entirely on the user's computer as a stand-alone software package, partly on the user's computer and partly on a remote computer or entirely on the remote computer or server. In the latter scenario, the remote computer may be connected in some embodiments to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry in order to perform aspects of the present disclosure.

Embodiments of the present disclosure as discussed herein may be implemented using a computing device illustrated in FIG. 2B. Referring now to FIG. 2B, FIG. 2B illustrates an embodiment of the present disclosure of the hardware configuration of a computing device 30 represents a hardware environment for practicing various embodiments of the present disclosure.

Computing device 30 has a processor 31 connected to various other components by system bus 32. An operating system 33 runs on processor 31 and provides control and coordinates the functions of the various components of FIG. 2B. An application 34 in accordance with the principles of the present disclosure runs in conjunction with operating system 33 and provides calls to operating system 33, where the calls implement the various functions or services to be performed by application 34. Application 34 may include, for example, a program for assessing a glioma in a subject, such as in connection with FIGS. 2A, 3, 4A-4B, 5A-5C, 6, 7A-7C8, and 8A-8B.

Referring again to FIG. 2B, read-only memory (“ROM”) 35 is connected to system bus 32 and includes a basic input/output system (“BIOS”) that controls certain basic functions of computing device 30. Random access memory (“RAM”) 36 and disk adapter 37 are also connected to system bus 32. It should be noted that software components including operating system 33 and application 34 may be loaded into RAM 36, which may be computing device's 30 main memory for execution. Disk adapter 37 may be an integrated drive electronics (“IDE”) adapter that communicates with a disk unit 38 (e.g., a disk drive). It is noted that the program for assessing a glioma in a subject, such as in connection with FIGS. 2A, 3, 4A-4B, 5A-5C, 6, 7A-7C8, and 8A-8B, may reside in disk unit 38 or in application 34.

Computing device 30 may further include a communications adapter 39 connected to bus 32. Communications adapter 39 interconnects bus 32 with an outside network (e.g., wide area network) to communicate with other devices.

Aspects of the present disclosure are described herein with reference to flowchart illustrations and/or block diagrams of methods, apparatus (systems), and computing devices according to embodiments of the disclosure. It will be understood that computer-readable program instructions can implement each block of the flowchart illustrations and/or block diagrams, and combinations of blocks in the flowchart illustrations and/or block diagrams.

These computer-readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein includes an article of manufacture including instructions which implement aspects of the function/act specified in the flowchart and/or block diagram block or blocks. The computer-readable program instructions may also be loaded onto a computer, other programmable data processing apparatus, or other devices to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computing devices according to various embodiments of the present disclosure. In this regard, each block in the flowchart or block diagrams may represent a module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Applications and Advantages

The methods and systems of the present disclosure provide numerous applications and advantages. For instance, in some embodiments, the methods and systems of the present disclosure may be utilized to prevent severe adverse effects of glioma treatments. In some embodiments, the methods and systems of the present disclosure may be utilized to reassess treatment strategies for patients that have a small tumor burden in a critical eloquent area.

In some embodiments, the methods and systems of the present disclosure could be utilized to predict treatment outcomes and provide clinicians (e.g., oncologists) with precise readouts for tumor burden and response to treatment. Such information could help guide therapeutic decisions, prevent unnecessary surgeries for pseudo progression, improve quality of life by preventing severe adverse effects of therapies, and help clinicians re-evaluate treatment strategies for certain patients (e.g., patients that have a small tumor burden in a critical eloquent area) to result in improved patient outcomes.

The methods and systems of the present disclosure can also provide critically needed criteria for rigorous monitoring of efficacy and toxicity of new therapeutic trials for gliomas (e.g., GBM). Additionally, the methods and systems of the present disclosure can enable the stratification and monitoring of patients diagnosed with gliomas (e.g., GBM) based on systemic miRNA/mRNA/protein profiles.

In some embodiments, the methods and systems of the present disclosure could be utilized to assess the efficacy of new glioma treatments. For instance, in some embodiments, the methods and systems of the present disclosure could be utilized as a component of GBM AGILE (i.e., Glioblastoma Adaptive Global Innovative Learning Environment). In some embodiments, the methods and systems of the present disclosure could be utilized to assess new potential treatments being tested to move the clinical trial process more rapidly, such as by rapidly assessing the toxicity of various treatments.

In some embodiments, the methods and systems of the present disclosure could be utilized to decrease the incidence of chemotherapy induced toxicity-related illnesses, as eliminating speculation would likely yield better patient outcomes. In some embodiments, the methods and systems of the present disclosure could be utilized to identify and monitor chemotherapy induced toxicity.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Correlation of Systemic miRNAs and Secreted Proteins to Tumor Burden and Treatment Induced Toxicities in GBM Patients

This example pertains to the identification of a set of systemic microRNAs (miRNAs) and secreted proteins that can be correlated to tumor burden and treatment induced toxicities in glioblastoma (GBM) patients. Through longitudinal analyses of patient plasma samples collected pre-and post-surgical resection of GBM tumors and during the course of currently available Standard of Care, irrespective of sex and age of patients as well as molecular and cellular heterogeneity of the tumor, Applicants have established: (1) specific systemic miRNAs and secreted proteins correlate with tumor burden and recurrence in GBM patients; and (2) specific systemic miRNAs and secreted proteins correlate with treatment induced toxicities in GBM patients.

After stringent statistical analyses, Applicants found the following miRNAs to be consistently and significantly altered despite age, sex as well molecular and cellular heterogeneity of the tumor: hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p, hu-mir-196b-5p, and hu-mir-151a-3p.

In particular, Applicants screened a list of miRNAs with z-scores that were used to determine the above as well as estimate that about 83% of patients have elevated or decreased post-surgical levels of specific miRNAs (correlating to tumor burden). The data, which were obtained from 27 patients, suggests that the difference of miRNA level from the analysis of blood draw between pre-and post-surgical resection is negative. This can be used as an indicator of early prediction of tumor growth and regrowth. Specifically, the effect size, as the difference on the scale of miRNA level, was −0.962. Translated onto the probability scale, Applicants estimate that about 83% of patients have elevated or decreased post-surgical levels of specific miRNAs (correlating to tumor burden). Taking the effect of size as the basis for power computation, Applicants predict that, with the suggested n=200 number of patients, Applicants can detect a statistically significant effect size of miRNA level as low as −0.168. This computation assumes that the significance level is 5% and the power of the test is 80%.

MiRNAs are molecular biomarkers that are small non-coding RNAs which function by regulating target gene expression post-transcriptionally. In GBMs, a characteristic miRNA expression pattern has been reported. Disease-associated miRNAs can be detected in blood cells and serum, specifically in GBM patients. Furthermore, precise miRNA signatures in blood are known to be associated with not only specific types of tumors but also with immune responses and suppression.

MiRNAs have the advantage of being able to cross the blood-brain barrier (BBB) and can easily be quantified. However, profiles of circulating miRNAs in GBM patients from time of diagnosis, post-surgical resection, through end of treatment and progression free survival or death have not been established.

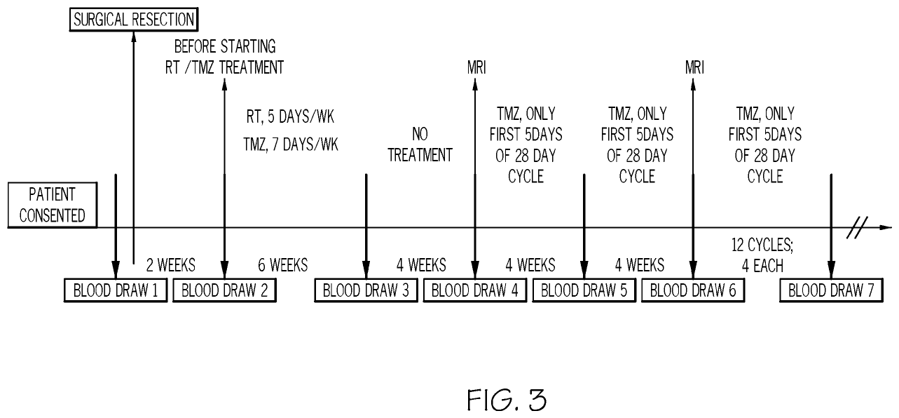

Building on experience gained from the recruitment, scheduling of SOC and clinical monitoring protocol for GBM patients at a single site clinical study, Applicants designed a pilot multi-center prospective clinical study wherein the time points for tissue and plasma collection were selected to coincide with scheduled SOC (FIG. 3). Plasma samples from a total of 36 GBM patients that met eligibility criteria at 4 sites were collected starting from pre-resection (Draw 1) for up to seven time points post-resection (Draw 2) and through SOC. Expression levels of miRNAs (from total exosomal RNA isolation) and secreted cytokines as well as growth factors were analyzed from six time points from 27 of these GBM patients (16 males and 11 females) and compared to plasma from 8 healthy donors (age matched ; 4 female and 4 male). These 27 patients were the cohort that had at least 6 blood draws.

### Example 1.1. Specific Systemic miRNAs Correlate With Tumor Burden and Recurrence in GBM Patients

Applicants'data demonstrate that irrespective of age, sex or mutational status, eleven miRNAs were expressed at levels correlating to tumor burden when compared across specific blood draws at different blood draw time points.

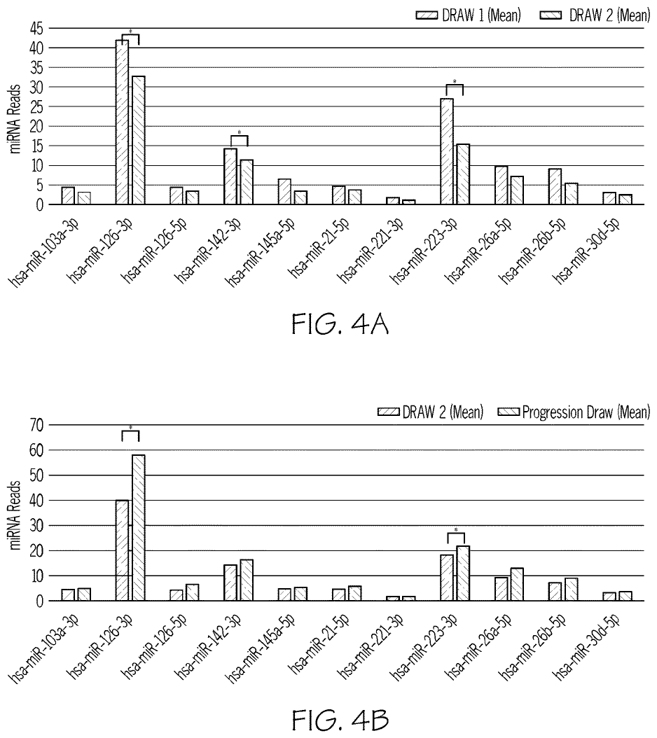

Applicants'data established that all 27 patients had significant decreases in expression levels of miR-126-3p, miR-142-3p, and miR-223-3p when comparing pre-to post-tumor resection (blood draws 1 vs draw 2; FIG. 4A). To further validate that these significant changes in miRNAs was associated with tumor burden, Applicants ran similar paired analyses with Draw 2 (that should have the lowest tumor burden after resection) versus all other draws collected in parallel with SOC (FIG. 4B). Applicants'rationale was that with GBM recurring in over 98% of patients within 7 to 10 months, the systemic levels of significant miRNAs (miR-126-3p, miR-142-3p, and miR-223-3p) that were elevated pre-resection but decreased post-resection should gradually start increasing again as tumors started recurring in patients.

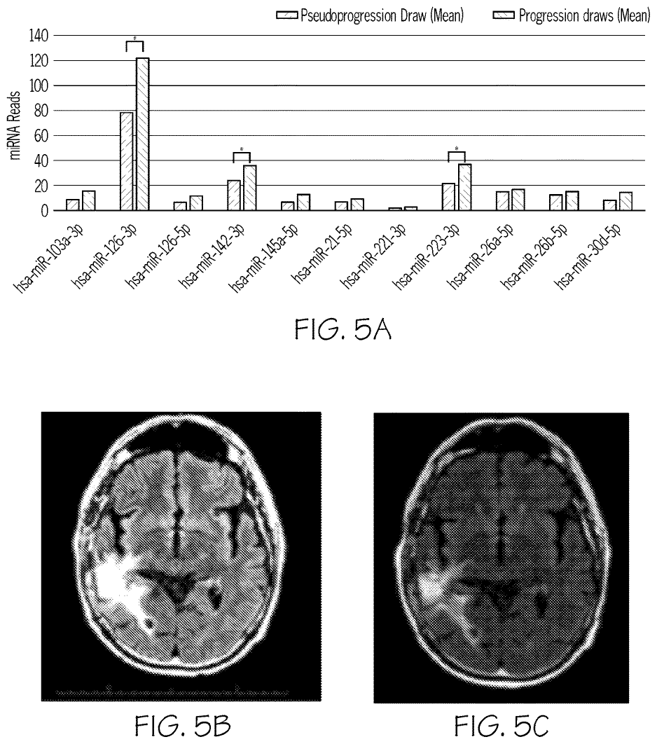

Along with running the paired analyses with Draw 2 (i.e. draws 2 vs draws 3, draws 2 vs draw 4, draws 1 vs draw 5, draws 2 vs draw 6, FIG. 5A), Applicants also reviewed all clinical data (specifically MRI reports) for all 27 patients (FIGS. 5B-5C). 25 patients demonstrated significant increases in expression levels of miR-126-3p, miR-142-3p, and miR-223-3p when comparing post-tumor resection to tumor progression (blood draws 2 vs draw 6 ; FIG. 5A). Draw 6 for two patient samples were degraded and could not be sequenced.

Furthermore, Applicants aimed to test hypotheses that miRNAs can track tumor burden by examining miRNA levels in patients that clinically were confirmed to have pseudo progression (by histological evaluation). Therefore, Applicants examined the miRNA levels in plasma samples taken from 2 patients from the original cohort of 36 that were recruited on this trial. These 2 patients had undergone a second resection due to the appearance of tumor growth on MRI. However, these lesions, post-surgical resection were confirmed by a surgical neuropathologist to not be tumor growth.

Applicants compared both their pre-surgery draws (before first and second resection). The data did not align (i.e., miRNAs) that were elevated before the first resection were not elevated at the time of the second resection despite the appearance of tumor on MRI. Histological confirmation of pseudo progression further validated Applicants'observation where Applicants did not observe any potential “tumor associated miRNA”.

Retrospectively, Applicants paired samples of known pseudo progression and tumor progression (FIGS. 5B-5C) time points of patient blood draws (FIG. 5A). These analyses provided Applicants with the same pattern of significant miRNAs as seen in FIG. 4B. The pseudo progression blood draws when compared to pre-resection (highest tumor burden) showed similar levels of miRNA expression as post-resection draws (with the lowest tumor burden).

All three comparisons between blood draws of differing tumor burdens showed a correlation of miRNA expression level to tumor burden for eleven miRNAs. As such, this Example has demonstrated that specific systemic microRNAs correlate with tumor burden and recurrence in GBM patients.

This finding marks a first in its nature to look at blood draws longitudinally for GBM patients throughout the entirety of their clinical treatment and show a potential biomarker for physicians to use in monitoring patient tumor progression. MiR-126-3p, miR-142-3p, and miR223-3p have statistically significant concentration profiles relative to their baseline miRNA concentration in the blood. Over 27 different GBM patients, the data demonstrates an increase in concentration of these three miRNAs that corresponds to an increase in tumor burden. Moreover, all three of these miRNAs have been shown to play unique roles in the inhibition (miR-223-3p), regulation (miR-142-3p), and sensitization (miR-126-3p) of GBM cell lines.

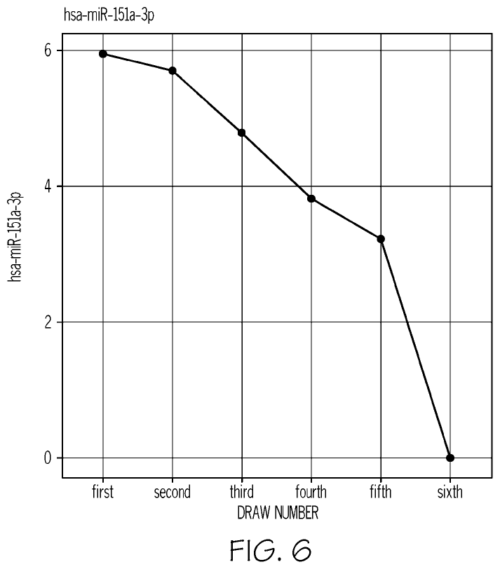

Additionally, Applicants observed in an initial analyses (of only 2 patients) a negative correlation between total miRNA content and chemotherapy induced toxicity levels. A significant decrease over the course of SOC and blood draws was observed in mir-151a-3p (FIG. 6). This decrease was observed in two patients that demonstrates treatment related toxicity. Moreover, mir-151a-30p has been shown to have a role in toxicity.

### Example 1.2. Specific Secreted Proteins in GBM Patient Plasma Demonstrate Significant Changes in Expression Levels During the Course of Standard of Care

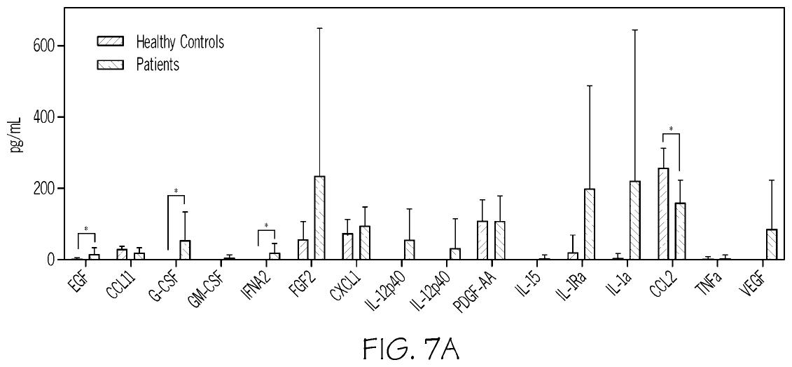

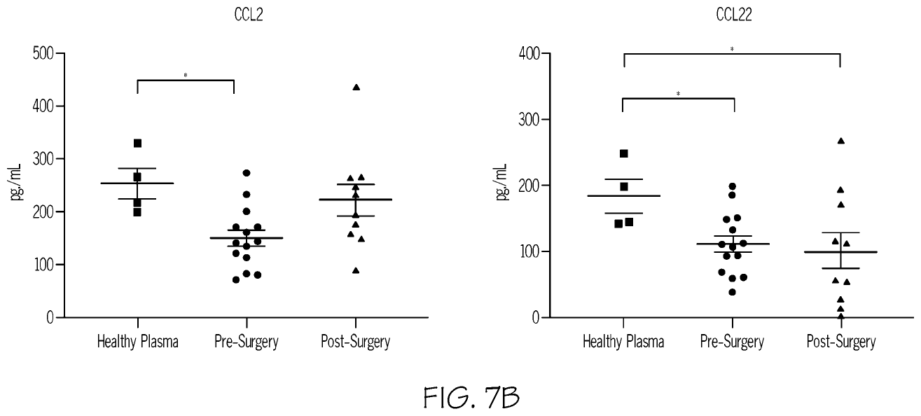

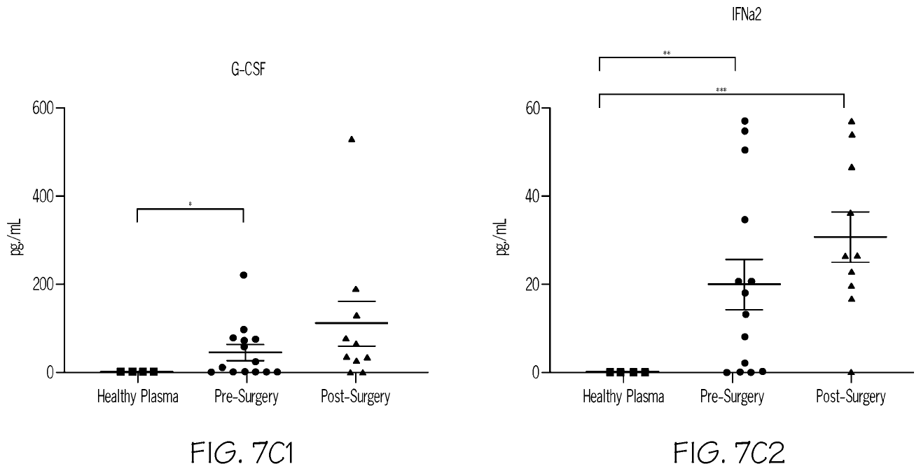

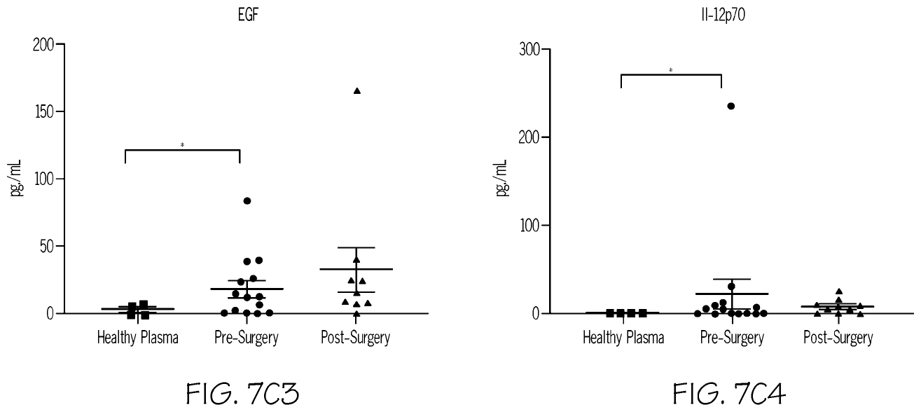

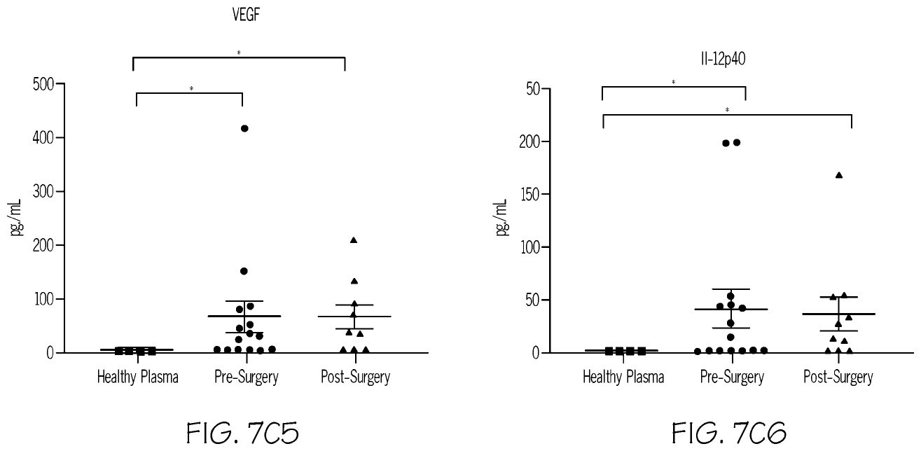

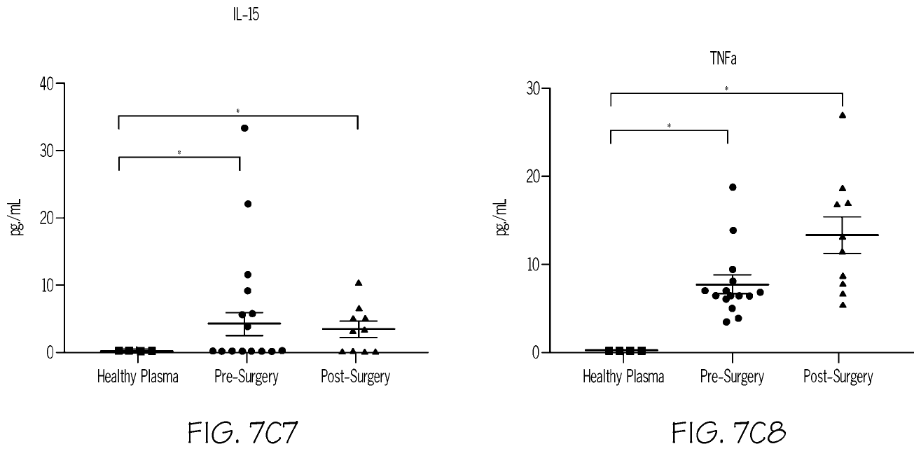

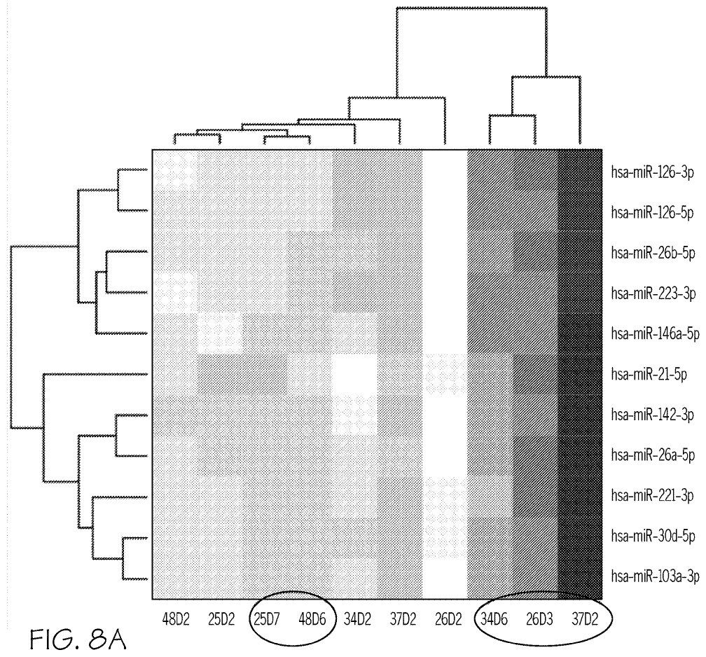

In addition to isolating and analyzing miRNAs in 27 GBM patients, Applicants also analyzed the plasma at 6 different time points for 48 specific cytokines, chemokines and growth factors (using the Human 48-Plex Luminex Assay). Healthy donor (age and sex matched) plasma were run as controls for baseline. From the initial 41 biomarkers that were tested in the Luminex assay, 4 of the factors were found to have a significance of less than p<0.05 when comparing patient pre-resection plasma samples to healthy control plasma samples (FIG. 7A), or to D2, draw taken immediately after tumor resection (lowest tumor burden) but before starting chemotherapy and/or radiation therapy (FIGS. 7B-7C8).

Applicants determined that there is a significant systemic decrease in CCL2 levels as well as a systemic increase in interferon-alpha 2(IFN-α2 ), granulocyte colony stimulating factor (G-CSF) and endothelial growth factor (EGF) levels compared to healthy controls, in paired patient pre-resection plasma samples. These results suggest that expression levels of specific proteins in the plasma may be suitable biomarkers for GBM tumor burden, and it provides insight into how GBM disrupt the immune response within the CNS and how the tumor burden manifests in the systemic setting across heterogenous patients. Additionally, as CCL2 is consistently down regulated across all patient samples that were tested, compared to controls, it may serve as a potential diagnostic marker and an indicator of the overall immune-suppressive state of the patient.

### Example 1.3. Additional Studies

Applicants also evaluated a total of 30 new patients (16 male and 14 female). Of the new patients, 17 showed real tumor progression and second resection. 2 showed pseudo progression.

The draws included: draw one (D1), which occurred before tumor resection, and which showed the highest tumor burden; draw two (D2), which occurred immediately after tumor resection, and which is correlated with lowest tumor burden; draw with progression (DProg), which was established after confirming progression in pathological diagnosis after second tumor resection; and draw with pseudo progression (DPseudo), which is established after confirming no progression in pathological diagnosis after second tumor resection.

A group of eleven miRNAs expression level changes across the three different draw comparisons (D1 vs D2//D2 vs DProg//DPseudo) were identified to be positively correlated to a change in tumor burden. Analyses between D1 and D2, D2 and DProg, and DPseudo and DProg draws demonstrate how these microRNAs have a directional change in blood concentration that is matched with patient tumor burden.

The Eleven miRNAs include miR-126-3p*, miR-126-5p, miR-146a-5p, miR-26b-5p, miR-223-3p*, miR-21-5p, miR-30d-5p, miR-103a-3p, miR-221-3p, miR-26a-5p, and miR142-3p*. “*” Indicates that the miRNAs differ significantly from all other miRNA differences within the patient averages.

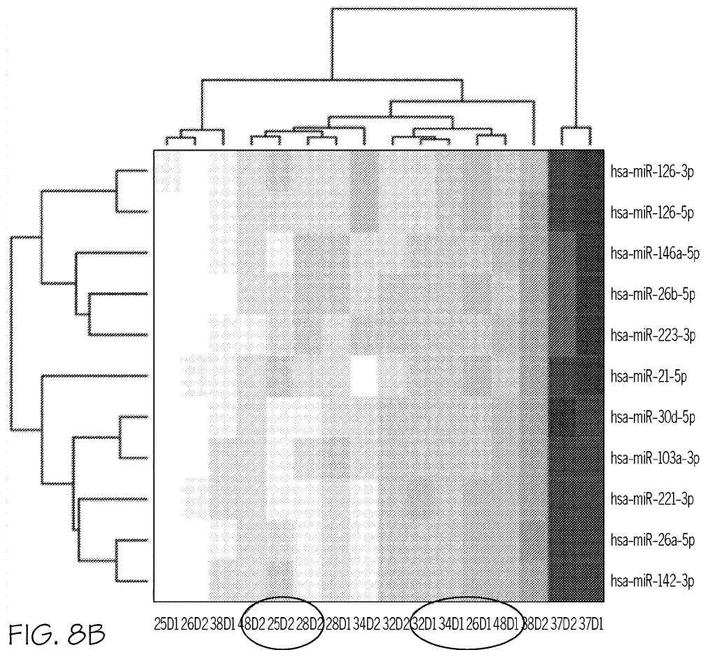

Specifically, all eleven microRNAs showed increased expression in D1 vs D2, DProg vs D2, and Dprog vs Dpseudo (FIGS. 8A-8B). Exosomal miR-126-3p, miR-142-3p, and miR-223-3p show highly significant changes in expression consistent with tumor burden cross all patients analyzed, which are optimal for establishing pseudo progression and hence preventing unnecessary second craniometry (FIGS. 8A-8B).

In summary, Applicants established comprehensive profiles of systemic biomarkers that correlate to tumor burden, response to treatment, as well as overall survival in GBM patients. Such biomarkers can be utilized as part of a CLIA-certified system that will be a minimally invasive and cost-effect complimentary platform to support currently available methods in the precise assessment of disease progression and treatment efficacy.

GBM has the highest annualized mean net costs per-patient for initial cost of care and for last-year-of-life care amongst any cancer group, with approximate costs of $150,000 and $175,000, respectively per patient. Applicants' findings could help prevent unnecessary surgeries for pseudo progression, improve quality of life by preventing severe adverse effects of therapies and help clinicians re-evaluate treatment strategies for patients that have a small tumor burden in a critical eloquent area and result in improved patient outcomes. Furthermore, Applicants'results could help enable the stratification and monitoring of patients diagnosed with GBM based on systemic miRNA/mRNA/protein profiles. Applicants'findings could also provide critically needed criteria for rigorous monitoring of efficacy and toxicity of all new therapeutic trials for GBM. Additionally, Applicants'findings could serve as the initial network for a universal system to monitor malignancies and treatments in patients with brain metastasis.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of assessing glioma in a subject, said method comprising:
receiving a plurality of measured biomarker levels of the subject, wherein the plurality of measured biomarker levels comprise one or more measured microRNA levels and one or more measured protein levels,
wherein the one or more measured microRNA levels comprise one or more measured levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p, hu-mir-196b-5p, hu-mir-151a-3p, or combinations thereof, and
wherein the one or more measured protein levels comprise one or more measured levels of CCL11, CCL2, CCL22, EGF, FGF-2, CXCL1, G-CSF, GM-CSF, IFN-α2, IL-6, IL-10, IL-12p40, IL-12p70, IL-15, IL-1Rα, IL-1α, TNFα, PDGF-AA, VEGF, or combinations thereof; and
assessing glioma in the subject based on the measured biomarker levels, wherein the assessing comprises correlating differentially expressed levels of the measured biomarkers to at least one of diagnosis of glioma, progression of glioma, treatment outcome, tumor burden, or combinations thereof.

2. The method of claim 1, wherein the glioma comprises glioblastoma (GBM).

3. The method of claim 1, wherein the assessing comprises correlating differentially expressed levels of the biomarkers to the progression of glioma in the subject, diagnosis of glioma in the subject, treatment outcome, tumor burden, or combinations thereof.

4-6. (canceled)

7. The method of claim 1,
wherein the one or more measured microRNA levels comprise measured levels of hu-mir-126-3p, hu-mir-mir-142-3p, and hu-mir-mir-223-3p,
wherein increased levels of hu-mir-126-3p, hu-mir-mir-142-3p, and hu-mir-mir-223-3p are correlated to a positive diagnosis of glioma, a positive progression of glioma, a negative treatment outcome, a positive tumor burden, or combinations thereof, and wherein decreased levels of hu-mir-126-3p, hu-mir-mir-142-3p, and hu-mir-mir-223-3p are correlated to a negative diagnosis of glioma, a negative progression of glioma, a positive treatment outcome, a negative tumor burden, or combinations thereof.

8. The method of claim 1, wherein the one or more measured microRNA levels comprise measured levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p, wherein increased levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p are correlated to a positive diagnosis of glioma, a positive progression of glioma, a negative treatment outcome, a positive tumor burden, or combinations thereof, and wherein decreased levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p are correlated to a negative diagnosis of glioma, a negative progression of glioma, a positive treatment outcome, a negative tumor burden, or combinations thereof.

9. The method of claim 1, wherein the one or more measured protein levels comprise measured levels of CCL2, CCL11, CCL22 and GM-CSF,
wherein decreased levels of CCL2, CCL11, CCL22 and GM-CSF are correlated to a positive diagnosis of glioma, a positive progression of glioma, a negative treatment outcome, a positive tumor burden, or combinations thereof, and
wherein increased levels of CCL2, CCL11, CCL22 and GM-CSF are correlated to a negative diagnosis of glioma, a negative progression of glioma, a positive treatment outcome, a negative tumor burden, or combinations thereof.

10. The method of claim 1, wherein the one or more measured protein levels comprise measured levels of IFN-α2, EGF, FGF-2, CXCL1, IL-12p40, IL-12p70, IL-15, IL-1Rα, IL-1α, TNFα, VEGF and G-CSF,
wherein increased levels of IFN-α2, EGF, FGF-2, CXCL1, IL-12p40, IL-12p70, IL-15, IL-1Rα, IL-1α, TNFα, VEGF and G-CSF are correlated to a positive diagnosis of glioma, a positive progression of glioma, a negative treatment outcome, a positive tumor burden, or combinations thereof, and
wherein decreased levels of IFN-α2, EGF, FGF-2, CXCL1, IL-12p40, IL-12p70, IL-15, IL-1Rα, IL-1α, TNFα, VEGF and G-CSF are correlated to a negative diagnosis of glioma, a negative progression of glioma, a positive treatment outcome, a negative tumor burden, or combinations thereof.

11. The method of claim 1, wherein the one or more measured protein levels comprise measured levels of G-CSF, IFNα2, EGF, IL-12p70, VEGF, IL-12p40, IL-15, and TNFα,
wherein increased levels of G-CSF, IFNα2, EGF, IL-12p70, VEGF, IL-12p40, IL-15, and TNFα are correlated to a positive diagnosis of glioma, a positive progression of glioma, a negative treatment outcome, a positive tumor burden, or combinations thereof, and
wherein decreased levels of G-CSF, IFNα2, EGF, IL-12p70, VEGF, IL-12p40, IL-15, and TNFα are correlated to a negative diagnosis of glioma, a negative progression of glioma, a positive treatment outcome, a negative tumor burden, or combinations thereof.

12. The method of claim 1, wherein the one or more measured protein levels comprise measured levels of CCL2 and CCL22,
wherein decreased levels of CCL2 and CCL22 are correlated to a positive diagnosis of glioma, a positive progression of glioma, a negative treatment outcome, a positive tumor burden, or combinations thereof, and
wherein increased levels of CCL2 and CCL22 are correlated to a negative diagnosis of glioma, a negative progression of glioma, a positive treatment outcome, a negative tumor burden, or combinations thereof.

13. The method of claim 1, wherein the subject is a human being suffering from glioma, glioblastoma (GBM), or combinations thereof.

14-15. (canceled)

16. The method of claim 1, wherein the subject has received treatment or is receiving treatment for glioma.

17. The method of claim 1, wherein the assessment occurs manually.

18. The method of claim 1, wherein the assessment occurs continuously.

19. The method of claim 1, wherein the assessment occurs automatically through the utilization of an algorithm, wherein the algorithm is a machine learning algorithm trained on the measured biomarkers.

20. (canceled)

21. The method of claim 1, further comprising a step of making a treatment decision based on the assessment.

22. The method of claim 21, wherein the treatment decision comprises monitoring the course of the glioma, removing the glioma from the subject, administering a therapeutic agent to the subject, modifying a pre-existing treatment regimen, or combinations thereof.

23. The method of claim 22, wherein the treatment decision comprises administering a therapeutic agent to the subject.

24. The method of claim 22, wherein the method is repeated after implementing the treatment decision.

25. The method of claim 1, wherein the differentially expressed levels of the measured biomarkers represent differentially expressed levels of the measured biomarkers in the subject relative to normal levels of the measured biomarkers, differentially expressed levels of the measured biomarkers in the subject after receiving treatment for glioma relative to the levels of the measured biomarkers in the subject prior to receiving treatment for glioma, or differentially expressed levels of the measured biomarkers in the subject after glioma tumor resection relative to the levels of measured biomarkers in the subject prior to glioma tumor resection.

26-27. (canceled)

28. A system for assessing glioma in a subject, wherein the system comprises a computing device, and wherein the computing device comprises:
programming instructions for receiving a plurality of measured biomarker levels of the subject, wherein the plurality of measured biomarker levels comprise one or more measured microRNA levels and one or more measured protein levels,
wherein the one or more measured microRNA levels comprise one or more measured levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p, hu-mir-196b-5p, hu-mir-151a-3p, or combinations thereof, and
wherein the one or more measured protein levels comprise one or more measured levels of CCL11, CCL2, CCL22, EGF, FGF-2, CXCL1, G-CSF, GM-CSF, IFN-α2, IL-6, IL-10, IL-12p40, IL-12p70, IL-15, IL-1Rα, IL-1α, TNFα, PDGF-AA, VEGF, or combinations thereof; and
programming instructions for assessing glioma in the subject based on the measured biomarker levels, wherein the assessing comprises correlating differentially expressed levels of the measured biomarkers to at least one of diagnosis of glioma, progression of glioma, treatment outcome, tumor burden, or combinations thereof.

29. The system of claim 28, wherein the computing device comprises an algorithm, and wherein the assessment occurs through the utilization of the algorithm.

30. The computing device of claim 28, wherein the algorithm is a machine learning algorithm trained on the measured biomarkers.

31. The system of claim 28, wherein the computing device further comprises programming instructions for making a treatment decision based on the assessment.

32. The system of claim 28, wherein the glioma comprises glioblastoma (GBM).

33. The system of claim 28, wherein the assessing comprises correlating differentially expressed levels of the biomarkers to the diagnosis of glioma in the subject, the progression of glioma in the subject, treatment outcome, tumor burden, or combinations thereof.

34-36. (canceled)

37. The system of claim 28,
wherein the one or more measured microRNA levels comprise measured levels of hu-mir-126-3p, hu-mir-mir-142-3p, and hu-mir-mir-223-3p,
wherein increased levels of hu-mir-126-3p, hu-mir-mir-142-3p, and hu-mir-mir-223-3p are correlated to a positive diagnosis of glioma, a positive progression of glioma, a negative treatment outcome, a positive tumor burden, or combinations thereof, and
wherein decreased levels of hu-mir-126-3p, hu-mir-mir-142-3p, and hu-mir-mir-223-3p are correlated to a negative diagnosis of glioma, a negative progression of glioma, a positive treatment outcome, a negative tumor burden, or combinations thereof.

38. The system of claim 28,
wherein the one or more measured microRNA levels comprise measured levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p,
wherein increased levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p are correlated to a positive diagnosis of glioma, a positive progression of glioma, a negative treatment outcome, a positive tumor burden, or combinations thereof, and
wherein decreased levels of hu-mir-103a-3p, hu-mir-146a-5p, hu-mir-2-1-5p, hu-mir-221-3p, hu-mir-26a-5p, hu-mir-26b-5p, hu-mir-30d-5p, hu-mir-126-3p, hu-mir-126-5p, hu-mir-142-3p, hu-mir-223-3p and hu-mir-196b-5p are correlated to a negative diagnosis of glioma, a negative progression of glioma, a positive treatment outcome, a negative tumor burden, or combinations thereof.

39. The system of claim 28, wherein the one or more measured protein levels comprise measured levels of CCL2, CCL11, CCL22 and GM-CSF,
wherein decreased levels of CCL2, CCL11, CCL22 and GM-CSF are correlated to a positive diagnosis of glioma, a positive progression of glioma, a negative treatment outcome, a positive tumor burden, or combinations thereof, and
wherein increased levels of CCL2, CCL11, CCL22 and GM-CSF are correlated to a negative diagnosis of glioma, a negative progression of glioma, a positive treatment outcome, a negative tumor burden, or combinations thereof.

40. The system of claim 28, wherein the one or more measured protein levels comprise measured levels of IFN-α2, EGF, FGF-2, CXCL1, IL-12p40, IL-12p70, IL-15, IL-1Rα, IL-1α, TNFα, VEGF and G-CSF,
wherein increased levels of IFN-α2, EGF, FGF-2, CXCL1, IL-12p40, IL-12p70, IL-15, IL-1Rα, IL-1α, TNFα, VEGF and G-CSF are correlated to a positive diagnosis of glioma, a positive progression of glioma, a negative treatment outcome, a positive tumor burden, or combinations thereof, and
wherein decreased levels of IFN-α2, EGF, FGF-2, CXCL1, IL-12p40, IL-12p70, IL-15, IL-1Rα, IL-1α, TNFα, VEGF and G-CSF are correlated to a negative diagnosis of glioma, a negative progression of glioma, a positive treatment outcome, a negative tumor burden, or combinations thereof.

41. The system of claim 28, wherein the one or more measured protein levels comprise measured levels of G-CSF, IFNα2, EGF, IL-12p70, VEGF, IL-12p40, IL-15, and TNFα,
wherein increased levels of G-CSF, IFNα2, EGF, IL-12p70, VEGF, IL-12p40, IL-15, and TNFα are correlated to a positive diagnosis of glioma, a positive progression of glioma, a negative treatment outcome, a positive tumor burden, or combinations thereof, and
wherein decreased levels of G-CSF, IFNα2, EGF, IL-12p70, VEGF, IL-12p40, IL-15, and TNFα are correlated to a negative diagnosis of glioma, a negative progression of glioma, a positive treatment outcome, a negative tumor burden, or combinations thereof.

42. The system of claim 28, wherein the one or more measured protein levels comprise measured levels of CCL2 and CCL22,
wherein decreased levels of CCL2 and CCL22 are correlated to a positive diagnosis of glioma, a positive progression of glioma, a negative treatment outcome, a positive tumor burden, or combinations thereof, and
wherein increased levels of CCL2 and CCL22 are correlated to a negative diagnosis of glioma, a negative progression of glioma, a positive treatment outcome, a negative tumor burden, or combinations thereof.

43. The system of claim 28, wherein the differentially expressed levels of the measured biomarkers represent differentially expressed levels of the measured biomarkers in the subject relative to normal levels of the measured biomarkers, differentially expressed levels of the measured biomarkers in the subject after receiving treatment for glioma relative to the levels of measured biomarkers in the subject prior to receiving treatment for glioma, differentially expressed levels of the measured biomarkers in the subject after glioma tumor resection relative to the levels of measured biomarkers in the subject prior to glioma tumor resection, or combinations thereof.

44-45. (canceled)
